(19) United States
(12) Patent Application Publication
(10) Pub. No.: US 2024/0252716 A1
(43) Pub. Date: Aug. 1, 2024

(54) SELF-ASSEMBLING NANOFIBROUS ULTRASHORT PEPTIDE HYDROGELS FOR VASCULAR TISSUE ENGINEERING

(71) Applicant: KING ABDULLAH UNIVERSITY OF SCIENCE AND TECHNOLOGY, Thuwal (SA)

(72) Inventors: Salwa Ahmed ALSHERI; Charlotte A.E. HAUSER, Thuwal (SA)

(21) Appl. No.: 18/565,702

(22) PCT Filed: May 30, 2022
(86) PCT No.: PCT/IB2022/055054

§ 371 Date: Nov. 30, 2023

Related U.S. Application Data
(60) Provisional application No. 63/196,792, filed on Jun. 4, 2021.

(51) Int. Cl. A61L 27/22; A61L 27/38; A61L 27/50; A61L 27/52

(52) U.S. Cl. CPC A61L 27/227; A61L 27/3808; A61L 27/507; A61L 27/52; A61L 2400/12

(57) ABSTRACT

The present disclosure relates generally to a tissue graft capable of undergoing angiogenesis, comprising at least one self-assemble peptide and at least one endothelial cells. The present disclosure further relates to a method of preparing such a tissue graft. The tube-like structure formed by endothelial cells within the graft can promote the growth and proliferation of other type of cell within the same 3D tissue graft and improve the result of tissue implantation.

### REFERENCE TO A “SEQUENCE LISTING”

The present application includes a Sequence Listing which has been submitted electronically in an ASCII text format. This Sequence Listing is named 114147-24072WO01_sequence listing.TXT was created on Jan. 18, 2022, is 1,045 bytes in size and is hereby incorporated by reference in its entirety.

### BACKGROUND

### Field of the Invention

The present disclosure relates generally to a tissue graft capable of undergoing angiogenesis, comprising at least one self-assemble peptide and at least one endothelial cells. The present disclosure further relates to a method of preparing such a tissue graft.

### Background of the Invention

The traditional approach to correct organ and tissue failure is the transplantation of healthy organs and tissues from donors. However, one of the major challenges of such an approach is organ and tissue rejection (Beyar, R., Challenges in organ transplantation. Rambam Maimonides medical journal 2011, 2 (2)). Subsequently, immunosuppressive therapy is required to prevent rejection. However, immunosuppression predisposes organ and tissue recipients to bacterial, viral, fungal, protozoal and parasitic infections (Patel, R.; Paya, C. V., Infections in solid-organ transplant recipients. Clinical microbiology reviews 1997,10 (1), 86-124). According to literature, such infections are a serious cause of morbidity and mortality in transplant patients (Duncan, M. D.; Wilkes, D. S., Transplant-related immunosuppression: a review of immunosuppression and pulmonary infections. Proceedings of the American Thoracic Society 2005, 2 (5), 449-455). Additionally, the shortage of donor organs is aggravating with time as a result of increasing organ failures, and population aging (Olson, J. L.; Atala, A.; Yoo, J. J., Tissue engineering: current strategies and future directions. Chonnam medical journal 2011, 47 (1), 1-13). In such a context, tissue engineering could be a promising solution (Rouchi, A. H.; Mandavi-Mazdeh, M., Regenerative medicine in organ and tissue transplantation: shortly and practically achievable? International journal of organ transplantation medicine 2015, 6 (3), 93). Injecting cells into damaged organs and tissues has been looked into for many years as a corrective measure. Nonetheless, this tissue engineering method is hindered by the lack of oxygen and nutrient delivery to the engineered construct, as well as instability of injected cells (Miklas, J. W.; Dallabrida, S. M.; Reis, L. A.; Ismail, N.; Rupnick, M.; Radisic, M., QHREDGS enhances tube formation, metabolism and survival of endothelial cells in collagen-chitosan hydrogels. PLoS One 2013, 8 (8), e72956; Rustad, K. C.; Sorkin, M.; Levi, B.; Longaker, M. T.; Gartner, G. C., Strategies for organ level tissue engineering. Organogenesis 2010, 6 (3), 151-157). Systemic vascularization depends essentially on arteries and veins which branch to form arterioles and venules that eventually end in capillary beds (Rouwkema, J.; Rivron, N. C.; van Blitterswijk, C. A., Vascularization in tissue engineering. Trends in biotechnology 2008, 26 (8), 434-441). Capillaries, therefore, represent the basic vascular unit.

The process by which new capillaries are formed in vivo is known as angiogenesis. This process includes complex interactions between the vascular endothelial cells and their surrounding microenvironment. During the process of angiogenesis, endothelial cells undergo a series of changes including proliferation, migration and cellular signaling to create a new capillary, under the influence of growth factors and environmental cues. Angiogenesis can be studied to provide insights into how endothelial cells behave in different environments to create vascular channels. This needs a scaffold that allows smooth cell-cell and cell-environment interactions. Therefore, artificial scaffolds are used in tissue engineering and regeneration.

One type of scaffold frequently used in the past is biological scaffold, such as gelatin, collagen, and hyaluronic acid (Gungor-Ozkerim, P. S.; Inci, I.; Zhang, Y. S.; Khademhosseini, A.; Dokmeci, M. R. Biomaterials Science 2018, 6, (5), 915-946). The use of biological scaffolds poses many challenges including the need for processing and the risk of hyper-stimulation of cellular components. Furthermore, biological scaffolds are not stable enough to support the growth and differentiation of the injected cells and often do not have an adequate level of stiffness. In addition to not providing good support for the embedded cells, the biological scaffold also poses additional challenge to the implementation of engineered tissue. For example, most of the soft porous biomaterials such as collagen are not able to endure fixation with suture because of their low tearing strength. Non-woven polyglycolic acid (PGA) fabrics that have frequently been used for scaffold fabrication are readily fixed by suturing but have such a high porosity that makes entrapment of sufficient amounts of cells difficult. ε-Caprolactone homopolymer also needs no reinforcement because of its excellent mechanical. However, because of the hydrophobic nature of it's surface and absence of functional groups that enable cell growth and proliferation, therefore, functionalization with other component and surface modification of ε-Caprolactone is needed to enhances the cellular activity.

In recent years, several scaffold models have been proposed to modulate angiogenesis. Most of which require the addition of growth factors to facilitate angiogenic signaling, or incorporate biological components such as tumor-derived basement membrane matrix gel (Matrigel®), or mammalian collagen. These biological components usually have weak mechanical strength, low shape fidelity, batch-to-batch variations and immunogenicity (Gjorevski, N.; Sachs, N.; Manfrin, A.; Giger, S.; Bragina, M. E.; Ordonez-Moran, P.; Clevers, H.; Lutolf, M. P. Nature 2016, 539, (7630), 560-564). Additionally, self-assembling peptide scaffolds have been reported. Such peptides undergo spontaneous assembly to form nanofibers of specific porosity and diameter. Moreover, utilizing self-assembled nanofibrous peptide hydrogels was proven to guarantee the localization of injected cells in a non-immunogenic, minimally invasive approach. In addition, due to their manageable mechanical and biological properties besides their biocompatibility and biodegradability, self-assembling peptide-based hydrogels are used nowadays as three-dimensional (3-D) scaffolds that closely resembles native extracellular matrices (ECMs) (Mizuguchi, Y.; Mashimo, Y.; Mie, M.; Kobatake, E., Temperature-Responsive Multifunctional Protein Hydrogels with Elastin-like Polypeptides for 3-D Angiogenesis. Biomacromolecules 2020, 21 (3), 1126-1135).

RADA16 is one of the most widely used self-assembling peptides for three-dimensional (3D) cell cultures. RATEA16 loading with the vascular endothelial growth factor (VEGF) was reported to support cell proliferation, migration, and tube formation of HUVECs (Zhang, R.; Liu, Y.; Qi, Y.; Zhao, Y.; Nie, G.; Wang, X.; Zheng, S. Self-assembled peptide hydrogel scaffolds with VEGF and BMP-2 Enhanced in vitro angiogenesis and osteogenesis. Oral Dis. 2021, DOI: 10.1111/odi.13785, in press). Due to its acidity, the pH of the self-assembled RADA16 hydrogel needs to be equilibrated to physiological pH prior to cell seeding or in vivo transplantation by immediately adding a large amount of media (Sun, Y.; Li, W.; Wu, X.; Zhang, N.; Zhang, Y.; Ouyang, S.; Song, X.; Fang, X.; Seeram, R.; Xue, W.; He, L.; Wu, W. Functional Self-Assembling Peptide Nanofiber Hydrogels Designed for Nerve Degeneration. ACS Appl. Mater. Interfaces 2016, 8, 2348-2359; Guo, J.; Su, H.; Zeng, Y.; Liang, Y.-X.; Wong, W. M.; Ellis-Behnke, R. G.; So, K.-F.; Wu, W. Reknitting the injured spinal cord by self-assembling peptide nanofiber scaffold. Nanomedicine 2007, 3, 311-321; Liu, X.; Wang, X.; Wang, X.; Ren, H.; He, J.; Qiao, L.; Cui, F.-Z. Functionalized self-assembling peptide nanofiber hydrogels mimic stem cell niche to control human adipose stem cell behavior in vitro. Acta Biomater. 2013, 9, 6798-6805). Furthermore, successful bone regeneration needs both good osteogenesis and vascularization, providing scaffolds that can support both osteogenic and angiogenic properties is much required. Therefore, an improved material that can support angiogenesis, ideally without the addition of bioactive components such as growth factor, is needed.

### SUMMARY

According to a first broad aspect the present disclosure provides a 3-dimensional tissue graft comprising: an ultrashort peptide scaffold; and at least one endothelial cell, wherein the endothelial cell forms network of tube-like structure.

According to a second broad aspect the present disclosure provides a method of creating 3-dimensional tissue graft comprising: dissolving at least one ultrashort peptide to form a peptide solution; constructing the tissue graft with the peptide solution; and seeding the endothelial cells on the tissue graft; wherein the ultrashort peptide is dissolved in water or buffer solution.

According to a third broad aspect the present disclosure provides a 3-dimensional tissue graft comprising: an ultrashort peptide scaffold, wherein the ultrashort peptide scaffold comprises at least one ultrashort peptide having a general formula selected from: AₙBₘX, BₘAₙX, XAₙBₘ and XBₘAₙ, wherein the total number of amino acids of the ultrashort peptide does not exceed 7 amino acids; wherein A is an aliphatic amino acids, selected from the group consisting of: isoleucine, leucine or any combination thereof, with n being an integer being selected from 0-5; wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3; wherein X is comprised of a polar amino acid, selected from the group consisting of: aspartic acid, glutamic acid, lysine, arginine, histidine, cysteine, serine, threonine, asparagine, and glutamine; and at least one endothelial cell, wherein the endothelial cell forms network of tube-like structure.

Other aspects and features of the present disclosure will become apparent to those skilled in the art upon review of the following description of specific embodiments of the invention in conjunction with the accompanying figures.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is SEM image showing nanofibers formation of IIFK (SEQ ID NO. 1) and IIZK (SEQ ID NO. 2) hydrogels according to an embodiment of the present disclosure.

FIG. 2 is TEM image showing nanofibers formation of IIFK (SEQ ID NO. 1) and IIZK (SEQ ID NO. 2) hydrogels according to an embodiment of the present disclosure.

FIG. 3 is a photo showing LIVE/DEAD staining of HUVEC cells cultured in the hydrogels and Matrigel® after different time of culture according to an embodiment of the present disclosure.

FIG. 4 is a photo showing attached cell after 4 hours of culture according to an embodiment of the present disclosure.

FIG. 5 is a graph showing the number of attached cell after 4 hours of culture according to an embodiment of the present disclosure.

FIG. 6 is a graph showing the ATP production by HUVEC cells in different scaffolds after 1 and 7 days of culture according to an embodiment of the present disclosure.

FIG. 7 is a photo showing the actin organization of HUVECs in IIFK (SEQ ID NO. 1) and IIZK (SEQ ID NO. 2) hydrogels and Matrigel® according to an embodiment of the present disclosure.

FIG. 8 is a photo showing the endothelial cell markers expression according to an exemplary embodiment of the present disclosure.

FIG. 9 is a bright-field images of HUVEC cells after 3 and 6 hours of culture showing the tube-like structure formed in IIFK (SEQ ID NO. 1) and IIZK (SEQ ID NO. 2) hydrogels and Matrigel® according to an exemplary embodiment of the present disclosure.

FIG. 10 is a graph showing the length of the tube-like structure form by HUVECs according to an exemplary embodiment of the present disclosure.

FIG. 11 is a graph showing the number of junctions of the tube-like structure form by HUVECs according to an exemplary embodiment of the present disclosure.

### DETAILED DESCRIPTION OF THE INVENTION

### Definitions

Where the definition of terms departs from the commonly used meaning of the term, applicant intends to utilize the definitions provided below, unless specifically indicated.

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present disclosure, the term “comprising”, the term “having”, the term “including,” and variations of these words are intended to be open-ended and mean that there may be additional elements other than the listed elements.

For purposes of the present disclosure, directional terms such as “top,” “bottom,” “upper,” “lower,” “above,” “below,” “left,” “right,” “horizontal,” “vertical,” “up,” “down,” etc., are used merely for convenience in describing the various embodiments of the present disclosure. The embodiments of the present disclosure may be oriented in various ways. For example, the diagrams, apparatuses, etc., shown in the drawing figures may be flipped over, rotated by 900 in any direction, reversed, etc.

For purposes of the present disclosure, a value or property is “based” on a particular value, property, the satisfaction of a condition, or other factor, if that value is derived by performing a mathematical calculation or logical decision using that value, property or other factor.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present disclosure, the term “amphiphilic” or “amphiphilicity” refers to being a compound consisting of molecules having a water-soluble group at one end and a water-insoluble group at the other end.

The term “aliphatic” means, unless otherwise stated, a straight or branched hydrocarbon chain, which may be saturated or mono- or poly-unsaturated and include heteroatoms. An unsaturated aliphatic group contains one or more double and/or triple bonds (alkenyl or alkynyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1 to 5, to 10, to 15 or to 20 carbon atoms. Examples of alkenyl radicals are straight-chain or branched hydrocarbon radicals which contain one or more double bonds. Alkenyl radicals generally contain about two to about twenty carbon atoms and one or more, for instance two, double bonds, such as about two to about ten carbon atoms, and one double bond. Alkynyl radicals normally contain about two to about twenty carbon atoms and one or more, for example two, triple bonds, preferably such as two to ten carbon atoms, and one triple bond. Examples of alkynyl radicals are straight-chain or branched hydrocarbon radicals which contain one or more triple bonds. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, tert-butyl, neopentyl, 3,3 dimethylbutyl. Both the main chain as well as the branches may furthermore contain heteroatoms as for instance N, O, S, Se or Si or carbon atoms may be replaced by these heteroatoms.

The term “heteroatom” as used herein means an atom of any element other than carbon or hydrogen.

For purposes of the present disclosure, the term “bioinks” as used herein means materials used to produce engineered/artificial live tissue, cellular grafts and organ substitutes (organoids) using 3D printing. In the present disclosure, these bioinks are mostly composed of hydrogel or organogel with cellular components embedded.

For purposes of the present disclosure, the term “gel”, and “nanogel” are used interchangeably. These terms refer to a is a network of polymer chains, entrapping water or other aqueous solutions, such as physiological buffers, of over 99% by weight. In an embodiment of the present disclosure, the polymer chains may be a peptide with repetitive sequences. If the self-assembly of the ultrashort peptides occurs in aqueous solution, hydrogels are formed. If organic solvents are used, organogels are formed.

For purposes of the present disclosure, the term “PBS” refers to a buffer solution commonly used in biological research, which is an abbreviation of phosphate-buffered saline. It is a water-based salt solution, helping to maintain a constant pH, as well as osmolarity and ion concentrations to match those of most cells. In some embodiments, PBS may include a water-based salt solution containing disodium hydrogen phosphate, sodium chloride and, in some formulations, potassium chloride and potassium dihydrogen phosphate.

For purposes of the present disclosure, the term “scaffolds” as used herein means the supramolecular network structures made from self-assembling ultra-short peptide or other polymer materials in the bioinks that provide support for the cellular components.

For purposes of the present disclosure, the term “structure fidelity” refers to the ability of 3D constructs to maintain its shape and internal structure over time.

For purposes of the present disclosure, the term “ultra-short peptide” refers to a sequence containing 3-7 amino acids. The peptides according an aspect of the present disclosure are also particularly useful for formulating aqueous or other solvent compositions, herein also sometimes referred to as “inks” or “bioinks” when mixed with cellular components, which may be used as inks for printing structures and as bioinks for printing cellular or tissue structures, in particular 3D structures. Such printed structures make use of the gelation properties of the peptides according to features of the present disclosure.

For purposes of the present disclosure, the terms “biocompatible” (which also can be referred to as “tissue compatible”) and “biocompatibility”, as used herein, refer to the property of a hydrogel that produces little if any adverse biological response when used in vivo.

For purposes of the present disclosure, the terms “v/v”, “v/v %” and “% v/v” are used interchangeably. These terms refer to Volume concentration of a solution.

For purposes of the present disclosure, the terms “w/v”, “w/v %” and “% w/v” are used interchangeably. These terms refer to Mass concentration of a solution, which is expressed as weight per volume.

### DESCRIPTION

Vascularization within biomaterial constructs requires growth, adhesion, and tube formation of endothelial cells in scaffolds. In a preferred embodiment, the endothelial cells are Human Umbilical Vein Endothelial Cells (HUVECs).

In one embodiment, the scaffolds are self-assembling nanofibrous ultrashort peptide hydrogels. The present disclosure provides ultrashort peptide sequences containing repetitive sequences capable of forming low molecular weight nanogels by self-assembly, wherein the ultrashort peptides are amphiphilic. The ultrashort peptides are able to self-assemble into supramolecular structures, having a composition of amino acids A, B, X, such as
AₙBₘX or BₘAₙX or XAₙBₘ or XBₘAₙ
   wherein the total number of amino acids of the ultrashort peptide does not exceed 7 amino acids;
   wherein A are comprised of aliphatic, i.e. non-aromatic, hydrophobic amino acids, selected from the group of aliphatic amino acids, such as isoleucine and leucine, with n being an integer being selected from 0-5;
   wherein B are comprised of one aromatic amino acid, such as tyrosine, tryptophan, or phenylalanine, preferably the hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3;
   wherein X is comprised of a polar amino acid, selected from the group of aspartic acid, glutamic acid, lysine, arginine, histidine, cysteine, serine, threonine, asparagine, and glutamine; and wherein when m=1, n>2.

In a preferred embodiment, the present disclosure provides ultrashort peptide sequences containing repetitive sequences capable of forming low molecular weight nanogels by self-assembly, wherein the ultrashort peptides are amphiphilic. The ultrashort peptides are able to self-assemble into supramolecular structures, having a composition of amino acids A, B, X, such as
AₙBₘX or BₘAₙX or XAₙBₘ or XBₘAₙ
   wherein the total number of amino acids of the ultrashort peptide does not exceed 7 amino acids;
   wherein A are comprised of aliphatic, i.e. non-aromatic, hydrophobic amino acids, selected from the group of aliphatic amino acids, such as isoleucine and leucine, with n being an integer being selected from 2-5;
   wherein B are comprised of one aromatic amino acid, such as tyrosine, tryptophan, or phenylalanine, preferably the hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 1 and 2; and
   wherein X is comprised of a polar amino acid, selected from the group of aspartic acid, glutamic acid, lysine, arginine, histidine, cysteine, serine, threonine, asparagine, and glutamine.

The amphiphilic peptide sequences containing repetitive sequences provided in the present disclosure show true supergelating properties, forming low molecular weight nanogels by entrapping water or other aqueous solutions, such as physiological buffers, of over 99% by weight. Therefore, hydrogels can be generated. These amphiphilic peptides have an innate propensity to self-assemble to 3D fibrous networks in form of hydrogels. These gels can also be termed nanogels, because the diameter of the single fibers of the gel's fiber network have nanometer diameters. These peptide compounds are self-driven by non-covalent interactions to form soft solid material. Based on the nature of the peptides involved, generally composed of natural amino acids, these soft materials can easily be used for biomedical applications, for tissue engineering, but also for technical applications.

It should be appreciated that the novel peptides have newly introduced aromatic amino acids in the hydrophobic part of the amphiphilic peptide structure. This is a significant improvement over prior peptides which focus solely on peptides containing aliphatic amino acids. The inclusion of aromatic amino acids is crucial for improving the self-assembly process over prior peptide configurations such as disclosed in WO 2011/123061 A1 which is incorporated herein by reference.

It should be appreciated that the novel peptides do not focus on the orientation of the hydrophobic part of the peptide compound as being limited to the N-terminus and the polar hydrophilic part limited to the C-terminus as is the case in prior peptides. The present amphiphilic peptides work well with having both orientations, as of N-terminus-hydrophobic part-hydrophilic part-C-terminus as well as N-terminus-hydrophili part-hydrophobi part-C-terminus.

### Self-Assembling Peptide Hydrogels Preparation and Characterization

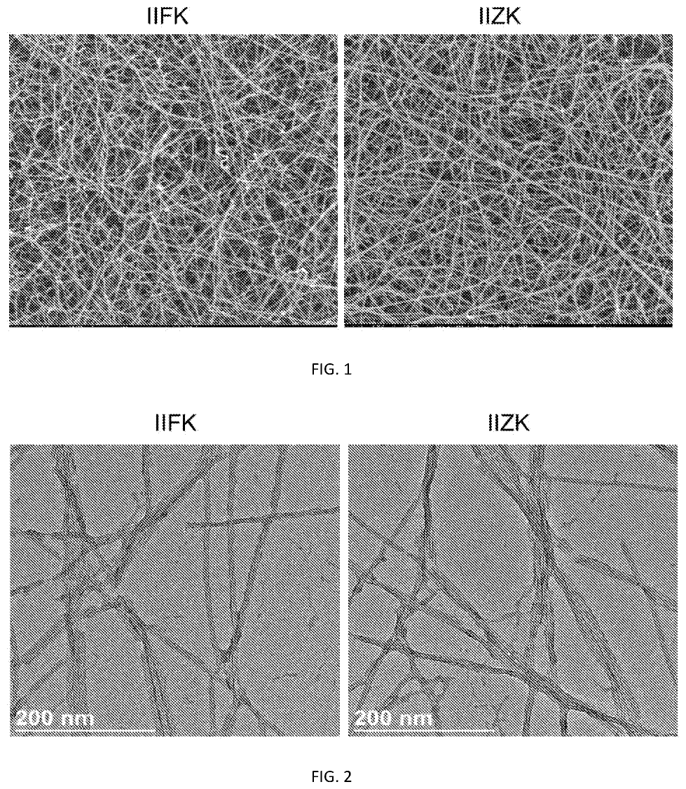

The solution-gel transition of both peptides (IIFK (SEQ ID NO. 1) and IIZK (SEQ ID NO. 2)) were induced by the addition of 1×PBS. Scanning electron microscopy (SEM) and transmission electron microscopy (TEM) examinations also confirmed the self-assembling nanofiber formations, as shown in FIGS. 1 and 2. The scale bars are 1 μm in FIGS. 1 and 200 nm in FIG. 2.

In one embodiment, these peptides form gels quickly at low concentration, which provide 3-D environment for cells embedded. Therefore, these hydrogels disclosed in the present disclosure should be useful for in vitro 3-D growth of cells.

In one embodiment, the stiffness of the peptide biomaterial in the present disclosure may be modulated, ranging from 1 kPa up to 270 kPa by adjusting the concentration of the peptide hydrogels, enabling control over mechanical factors. In vivo cells reside in 3D niches, in which different factors, such as mechanical cues, interact and play an essential role in cell function and fate.¹⁶ In addition, the mechanical strength is also an important consideration of manipulations of implantation, such as suturing.

### Cell Viability and Attachment

Viability of HUVEC cells were evaluated by using LIVE/DEAD® staining at different time points in order to assess the compatibility of peptide scaffolds with cells. In one embodiment, the viability of HUVEC cells was determined after 24 h, 4 days and 7 days of culturing within peptide scaffolds.

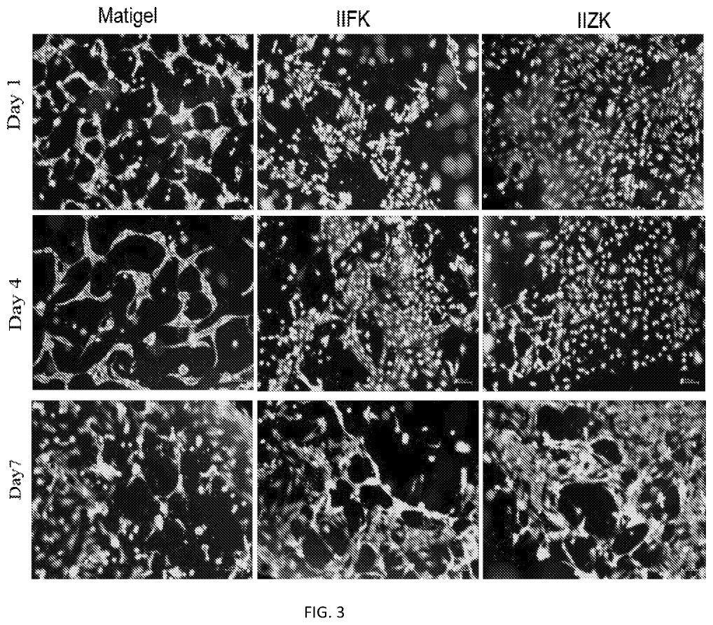

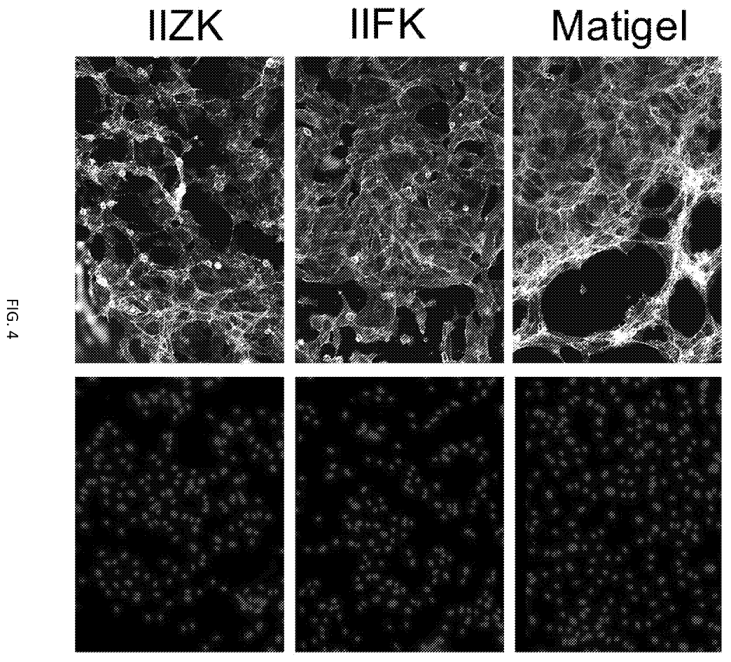

In one embodiment, cells grown in IIFK (SEQ ID NO. 1) and IIZK (SEQ ID NO. 2) hydrogels have shown a remarkable growth comparing to the Matrigel® with low dead cells as shown in FIG. 3, indicated by greater number of living cells stained in green in IIFK (SEQ ID NO. 1) and IIZK (SEQ ID NO. 2) hydrogels. In FIG. 3, for living cells were stained in green by calcein AM, while dead cells were stained in red by ethidium homodimer (EthD-1). The higher viability of HUVEC cells in IIFK (SEQ ID NO. 1) and IIZK (SEQ ID NO. 2) hydrogels than in Matrigel® indicates the biocompatibility of the peptide scaffolds.

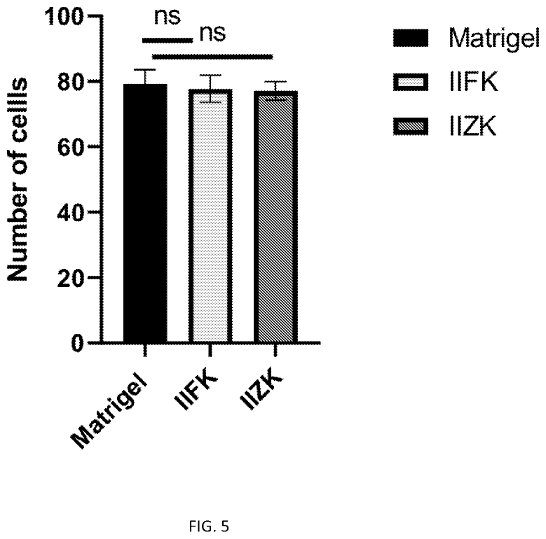

In another embodiment, HUVEC cells cultured in IIFK (SEQ ID NO. 1) and IIZK (SEQ ID NO. 2) hydrogels have comparable level of cell attachment to HUVEC cells cultured in Matrigel®. Cell attachment was evaluated by seeding HUVECs on top of coverslips coated with Matrigel® (control) and different hydrogels. After incubation for 4 hours, efficient cell extension and adhesion in both control and hydrogels can be clearly seen, and also tube-like structure can be observed, as shown in FIG. 4. Furthermore, the number of cells attached was calculated for different scaffold used. As shown in FIG. 5, there is no significant difference in cell attachment between the IIFK (SEQ ID NO. 1) and IIZK (SEQ ID NO. 2) hydrogels and Matrigel®, which indicates that both peptides hydrogels support the attachment of HUVEC cells.

### Cell Proliferation

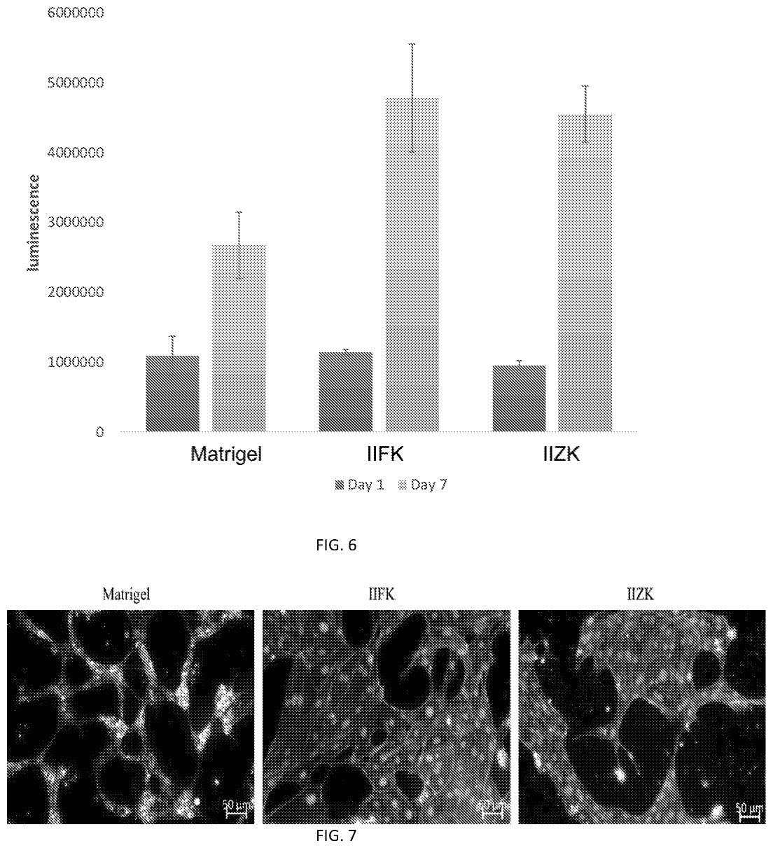

In one embodiment, HUVEC cells have higher proliferation cultured in peptides than in Matrigel®. Proliferation of cells cultured in both hydrogels and Matrigel® was evaluated by measuring the production of ATP after culturing for different period of time. The differences in ATP produced when cultured in different scaffolds was calculated. As shown in FIG. 6, there is no significant difference between IIFK (SEQ ID NO. 1), IIZK (SEQ ID NO. 2), and Matrigel® after 24 h of culture. However, the amount of ATP produced was significantly higher in IIFK (SEQ ID NO. 1) and IIZK (SEQ ID NO. 2) hydrogels, compared Matrigel® after 7 day of culture as also shown in FIG. 6.

### Cell Morphology and Functionality

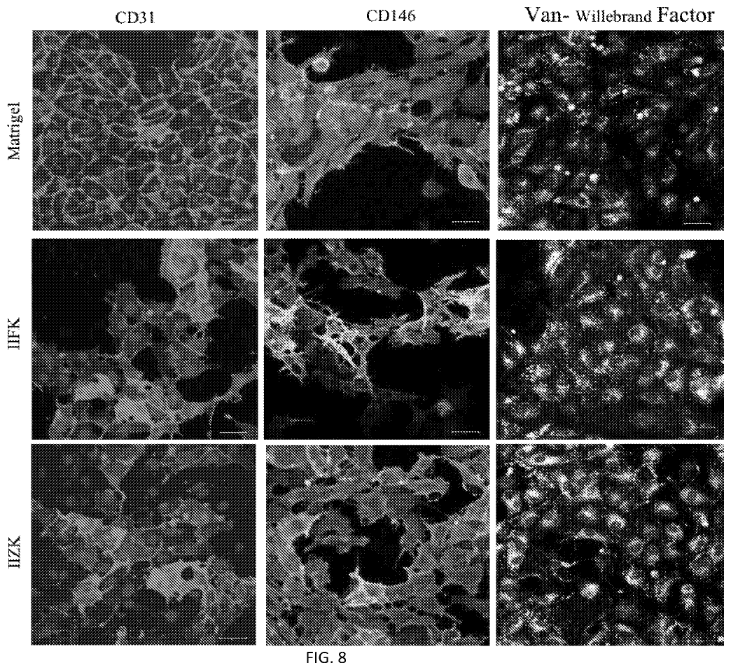

In one embodiment, HUVECs grown in peptide scaffolds maintain their normal morphology and functionality. Cell morphology can be suggested by the cytoskeleton organization of HUVECs. In the present disclosure, the cytoskeleton organization of HUVECs grown in the nanofibers scaffolds was visualized by staining the actin of cells with rhodamine-phalloidin. Cell morphology and the organization of actin structures of HUVECs grown in different scaffolds were compared and shown in FIG. 7. Cells grown in IIFK (SEQ ID NO. 1) and IIZK (SEQ ID NO. 2) peptide hydrogels, displayed a fibroblastic morphology of HUVECs, which exhibiting a high level of spreading with a well-stretching actin fiber.

The functionality of HUVECs in different scaffolds was determined by the expression of universal endothelial cell markers, including CD34, CD146 and Von-Willebrand factor (vWF). As shown in FIG. 8, all three markers are well expressed in IIFK (SEQ ID NO. 1) and IIZK (SEQ ID NO. 2) hydrogels similar to that in Matrigel®.

### Tube Like Structure Formation by HUVEC Cells

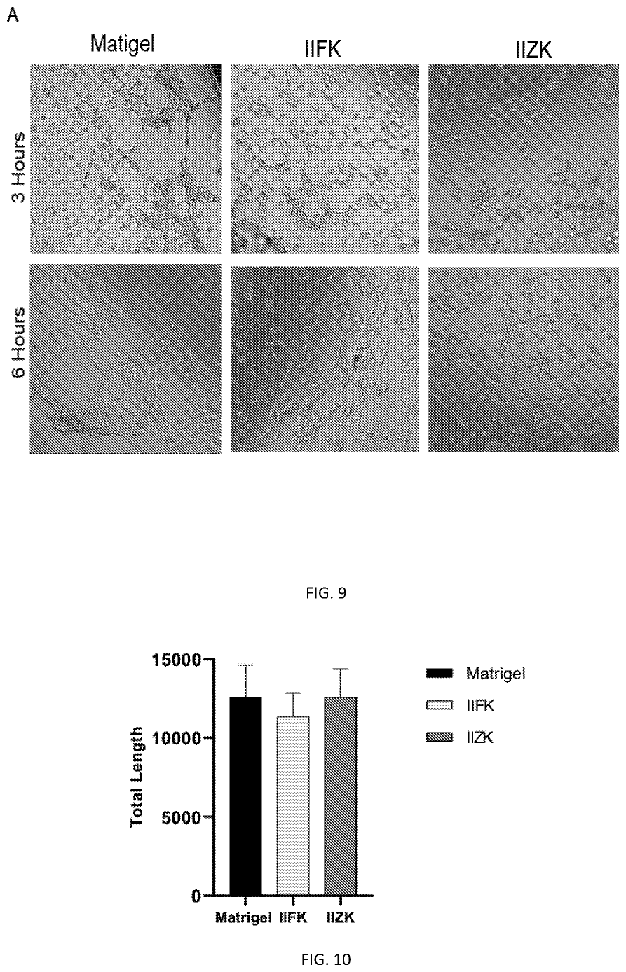

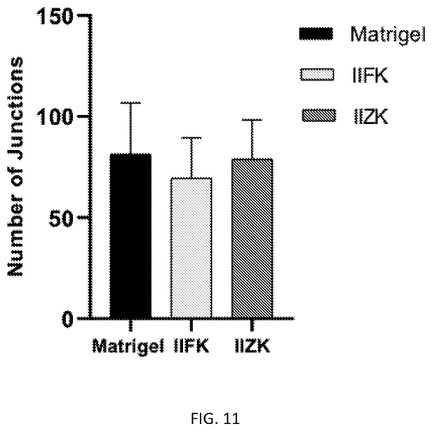

In one embodiment, HUVEC cells form tube-like structures when cultured in peptide hydrogels. In determining the effects of peptide scaffolds in facilitating the formation of the capillary-like structure formed by HUVEC cells, there was no addition of growth factors, after cells were plated on the surfaces of IIFK (SEQ ID NO. 1) and IIZK (SEQ ID NO. 2) scaffolds and Matrigel® (control). The typical morphologies of cell organizations after 2-6 hours of culture are shown in FIG. 9. It can be clearly seen in FIG. 9 that HUVECs formed capillary-like structure networks on peptide and Matrigel®. In one embodiment, HUVECs seeded in peptide scaffold formed network with capillary-like structure 4 hours after seeding, which was comparable to cell behaviors in Matrigel®. However, Matrigel® scaffold contains several growth factors that stimulate capillary-like structure formation rapidly. In another embodiment, the length and number of junctions of tube-like structure formed by HUVECs in peptide hydrogels without growth factor are also comparable to those in Matrigel® with the addition of several growth factors. Tube length and diameter were evaluated using ImageJ software. As shown in FIG. 10, there is no significant difference between Matrigel® and peptides IIFK (SEQ ID NO. 1) and IIZK (SEQ ID NO. 2). In our study, no growth factors were added to the peptide scaffold. In addition, as shown in FIG. 11, the number of junctions formed by HUVECs in IIFK (SEQ ID NO. 1) and IIZK (SEQ ID NO. 2) hydrogels without growth factor is also comparable to that in Matrigel® with growth factors. This result suggests that the peptides can stimulate the capillary-like structure formation and this may play an important role in angiogenesis.

Having described the many embodiments of the present disclosure in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

### EXAMPLES

### Example 1

### Peptide Hydrogel Preparation

The tetramer self-assembling peptides Ac-IIZK (SEQ ID NO. 2)-NH₂ (Ac-Ile-Val-Cha-Lys-NH₂) and Ac-IIFK (SEQ ID NO. 1)-NH₂ (Ac-Ile-Val-Phe-Lys-N H₂) were manually synthesized by using solid phase peptide synthesis and purified to above 95% via HPLC. Amino acid and peptide content analysis were performed. Lyophilized peptide was dissolved in milliQ® water and vortexed to get a homogenous solution. Subsequently, 10×phosphate buffered saline at the final concentration of 1× was added to the peptide solution and vortexed shortly. Gelation occurred within few seconds in IIZK (SEQ ID NO. 2) at I mg/mL and IIFK (SEQ ID NO. 1) at 2 mg/mL peptide concentration.

### Example 2

### Cell Culture

Human Umbilical Venous Endothelial Cells (HUVECs, CC-2517) was purchased from Lonza®. Cells were cultured in medium EGM-2 (Lonza®). The cells were maintained either in a T125 or T75 cell culture flask (Corning®, USA) at 37° C. in a incubator with 95% air and 5% CO₂. The cells were subcultured by trypsin at approximately 80% confluence. The culture media was replenished every 48 hours.

### Example 3

### Live/Dead Staining

HUVEC cells were seeded and treated with peptides according to the protocol described above. After 24 h of incubation, the media was removed and replaced with DPBS solution containing approximately 2 mM calcein AM and 4 mM ethidium homodimer-1 (LIVE/DEAD® Viability/Cytotoxicity Kit, Life Technologies®) and incubated for 30 min in dark. Before imaging, the staining solution was removed and fresh DPBS was added. Stained cells were imaged with ZEISS® fluorescent microscope.

### Example 4

### Cytoskeletal Staining and Endothelial Cell Markers

Immunostaining was performed after 24 h of culture. In brief, the cells were fixed with 4% paraformaldehyde solution for 30 minutes and incubated in a cold cytoskeleton buffer (3 mM MgCl₂, 300 mM sucrose and 0.5% Triton X-100 in PBS solution) for 10 minutes to permeabilize the cell membranes. The permeabilised cells were incubated in blocking buffer solution (5% FBS, 0.1% Tween-20, and 0.02% sodium azide in PBS) for 30 minutes at 37° C., followed by incubation with rhodamine-phalloidin (1:300) for 1 hour at 37° C. In case of endothelial cell markers, the sample incubated with Mouse anti-Endothelial Cell CD146 ( 1/500), Rabbit anti-VWF ( 1/5000) or Mouse anti-CD31 ( 1/50) for 1 hour and followed by secondary antibody. Furthermore, the cells were incubated in DAPI for 5 minutes at room temperature to counterstain the nucleus. Finally, the cells were observed and imaged using a laser scanning confocal microscope (Zeiss® LSM 710 Inverted Confocal Microscope, Germany).

### Example 5

### 3D Culture of Cells in Peptide Hydrogels

HUVEC cells were encapsulated in peptide hydrogels in 96 well tissue culture plates. Peptide solution was added to the plate at 40 μL per well. HUVEC cells suspended in 2×PBS were added to each well at 40,000 cells/well and gently mixed. The final concentration of the peptide hydrogel was 1× after the addition of 2×PBS containing cells. Gelation occurred within 3-5 minutes and subsequently, the culture medium was added to the wells. At pre-determined time points, the 3D cell viability assay, live/dead assay and florescence staining were performed.

### Example 6

### 3D Cell Proliferation Assay

The CellTiter-Glo® luminescent 3D cell viability assay is a method to determine the number of viable cells in 3D hydrogels based on quantification of the ATP present, which signals the presence of metabolically active cells. After each time point, an equal amount of CellTiter-Glo® luminescent reagent was added to the same amount of media in each well. The contents were mixed for 5 minutes to digest the hydrogels and then incubated for 10 minutes. After incubation, the luminescence was recorded using a plate reader (PHERAstar® FS, Germany).

### Example 7

### In Vitro Angiogenesis Study

Peptide hydrogel or Matrigel® were placed in 24 well plate, and human umbilical vein endothelial cells (HUVECs) 40,000 cells/well were added on top of peptide gel or Matrigel®. The amount of solutions in each well is about 200 μl. Cells were cultured in Endothelial growth media for 24 hours. Cells were then investigated using inverted microscope, and images were analyzed by Image J using Angiogenesis Analyzer.

### Example 8

### Statistical Analysis

All experimental approaches were executed in triplicates. Results are represented as mean±standard deviation, n≥3. The differences observed in HUVECs behavior in different scaffolds were compared and statistical analysis was analyzed using a student's t-test, and values with p<0.05 was considered to be statistically significant.

It is intended that the invention not be limited to the particular embodiment disclosed herein contemplated for carrying out this invention, but that the invention will include all embodiments falling within the scope of the claims.

All documents, patents, journal articles and other materials cited in the present application are incorporated herein by reference.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

### REFERENCES

The following references are referred to above and are incorporated herein by reference:
1. Beyar, R., Challenges in organ transplantation. Rambam Maimonides medical journal 2011, 2 (2).
2. Patel, R.; Paya, C. V., Infections in solid-organ transplant recipients. Clinical microbiology reviews 1997,10 (1), 86-124.
3. Duncan, M. D.; Wilkes, D. S., Transplant-related immunosuppression: a review of immunosuppression and pulmonary infections. Proceedings of the American Thoracic Society 2005, 2 (5), 449-455.
4. Olson, J. L.; Atala, A.; Yoo, J. J., Tissue engineering: current strategies and future directions. Chonnam medical journal 2011, 47 (1), 1-13.
5. Rouchi, A. H.; Mandavi-Mazdeh, M., Regenerative medicine in organ and tissue transplantation: shortly and practically achievable? International journal of organ transplantation medicine 2015, 6 (3), 93.
6. Miklas, J. W.; Dallabrida, S. M.; Reis, L. A.; Ismail, N.; Rupnick, M.; Radisic, M., QHREDGS enhances tube formation, metabolism and survival of endothelial cells in collagen-chitosan hydrogels. PLoS One 2013, 8 (8), e72956.
7. Rustad, K. C.; Sorkin, M.; Levi, B.; Longaker, M. T.; Gartner, G. C., Strategies for organ level tissue engineering. Organogenesis 2010, 6 (3), 151-157.
8. Rouwkema, J.; Rivron, N. C.; van Blitterswijk, C. A., Vascularization in tissue engineering. Trends in biotechnology 2008, 26 (8), 434-441.
9. Mizuguchi, Y.; Mashimo, Y.; Mie, M.; Kobatake, E., Temperature-Responsive Multifunctional Protein Hydrogels with Elastin-like Polypeptides for 3-D Angiogenesis. Biomacromolecules 2020, 21 (3), 1126-1135.
10. Gjorevski, N.; Sachs, N.; Manfrin, A.; Giger, S.; Bragina, M. E.; Ordonez-Moran, P.; Clevers, H.; Lutolf, M. P. Nature 2016, 539, (7630), 560-564.
11. Gungor-Ozkerim, P. S.; Inci, I.; Zhang, Y. S.; Khademhosseini, A.; Dokmeci, M. R. Biomaterials Science 2018, 6, (5), 915-946.
12. Zhang, R.; Liu, Y.; Qi, Y.; Zhao, Y.; Nie, G.; Wang, X.; Zheng, S. Self-assembled peptide hydrogel scaffolds with VEGF and BMP-2 Enhanced in vitro angiogenesis and osteogenesis. Oral Dis. 2021, DOT: 10.1111/odi.13785, in press.
13. Sun, Y.; Li, W.; Wu, X.; Zhang, N.; Zhang, Y.; Ouyang, S.; Song, X.; Fang, X.; Seeram, R.; Xue, W.; He, L.; Wu, W. Functional Self-Assembling Peptide Nanofiber Hydrogels Designed for Nerve Degeneration. ACS Appl. Mater. Interfaces 2016, 8, 2348-2359.
14. Guo, J.; Su, H.; Zeng, Y.; Liang, Y.-X.; Wong, W. M.; Ellis-Behnke, R. G.; So, K.-F.; Wu, W. Reknitting the injured spinal cord by self-assembling peptide nanofiber scaffold. Nanomedicine 2007, 3, 311-321.
15. Liu, X.; Wang, X.; Wang, X.; Ren, H.; He, J.; Qiao, L.; Cui, F.-Z. Functionalized self-assembling peptide nanofiber hydrogels mimic stem cell niche to control human adipose stem cell behavior in vitro. Acta Biomater. 2013, 9, 6798-6805.
16. Discher, D. E.; Mooney, D. J.; Zandstra, P. W. Science 2009, 324, (5935), 1673-1677.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, products specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

While the present disclosure has been disclosed with references to certain embodiments, numerous modifications, alterations, and changes to the described embodiments are possible without departing from the sphere and scope of the present disclosure, as defined in the appended claims. Accordingly, it is intended that the present disclosure is not limited to the described embodiments, but that it has the full scope defined by the language of the following claims, and equivalents thereof.

1. A 3-dimensional tissue graft comprising:
an ultrashort peptide scaffold; and
at least one endothelial cell,
wherein the endothelial cell forms network of tube-like structures.

2. The 3-dimensional tissue graft of claim 1, wherein the ultrashort peptide scaffold comprises at least one ultrashort peptide having a general formula selected from:
AₙBₘX, BₘAₙX, XAₙBₘ and XBₘAₙ
wherein the total number of amino acids of the ultrashort peptide does not exceed 7 amino acids;
wherein A is an aliphatic amino acids, selected from the group consisting of: isoleucine, leucine or any combination thereof, with n being an integer being selected from 0-5;
wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3; and
wherein X is comprised of a polar amino acid, selected from the group consisting of: aspartic acid, glutamic acid, lysine, arginine, histidine, cysteine, serine, threonine, asparagine, and glutamine.

3. The 3-dimensional tissue graft of claim 1, wherein the ultrashort peptide scaffold comprises at least one selected from the group consisting of IIFK (SEQ ID NO. 1), IIZK (SEQ ID NO. 2), KFII (SEQ ID NO. 3) and KZII (SEQ ID NO. 4), wherein I is isoleucine, F is phenylalanine, K is lysine, and Z is cyclohexylalanine.

4. The 3-dimensional tissue graft of claim 1, wherein the ultrashort peptide further comprises an N-terminal protecting group and a C-terminal protecting group.

5. The 3-dimensional tissue graft of claim 4, wherein the N-terminal protecting group is an acetylated group and the C-terminal protecting group is an amidated group.

6. The 3-dimensional tissue graft of claim 1, wherein the concentration of ultrashort self-assembling peptide used to form the scaffolds is 1 mg/ml-8 mg/ml.

7. The 3-dimensional tissue graft of claim 1, wherein the concentration of IIZK is 1 mg/ml and the concentration of IIFK is 2 mg/ml.

8. The 3-dimensional tissue graft of claim 1, wherein the stiffness of the peptide scaffolds is 1-270 kPa.

9. The 3-dimensional tissue graft of claim 1, wherein the endothelial cell is Human Umbilical Vein Endothelial Cells (HUVEC).

10. The 3-dimensional tissue graft of claim 1, wherein the density of the endothelial cells within the graft is at least 40,000 per 200 μl culture.

11. The 3-dimensional tissue graft of claim 1, wherein the viability of endothelial cells is at least 90%.

12. The 3-dimensional tissue graft of claim 1, wherein the tissue graft is free of angiogenesis-inducing growth factors.

13. A method of creating 3-dimensional tissue graft comprising:
dissolving at least one ultrashort peptide to form a peptide solution;
mixing the endothelial cells with the peptide solution; and
constructing the tissue graft with the peptide solution;
wherein the ultrashort peptide is dissolved in water or buffer solution.

14. A method of creating 3-dimensional tissue graft comprising:
dissolving at least one ultrashort peptide to form a peptide solution;
constructing the tissue graft with the peptide solution; and
seeding the endothelial cells on the tissue graft;
wherein the ultrashort peptide is dissolved in water or buffer solution.

15. The method of claim 14, wherein the ultrashort peptide scaffold comprises at least one ultrashort peptide having a general formula selected from:
AₙBₘX, BₘAₙX, XAₙBₘ and XBₘAₙ
wherein the total number of amino acids of the ultrashort peptide does not exceed 7 amino acids;
wherein A is an aliphatic amino acids, selected from the group consisting of: isoleucine, leucine or any combination thereof, with n being an integer being selected from 0-5;
wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3;
wherein X is comprised of a polar amino acid, selected from the group consisting of: aspartic acid, glutamic acid, lysine, arginine, histidine, cysteine, serine, threonine, asparagine, and glutamine.

16. The method of claim 14, wherein the ultrashort peptide scaffold comprises at least one selected from the group consisting of IIFK (SEQ ID NO. 1), IIZK (SEQ ID NO. 2), KFII (SEQ ID NO. 3) and KZII (SEQ ID NO. 4), wherein I is isoleucine, F is phenylalanine, K is lysine, and Z is cyclohexylalanine.

17. The method of claim 14, wherein the ultrashort peptide further comprises an N-terminal protecting group and a C-terminal protecting group.

18. The method of claim 17, wherein the N-terminal protecting group is an acetylated group and the C-terminal protecting group is an amidated group.

19. The method of claim 14, wherein the concentration of the peptide solution is 0.1 mg/ml-100 mg/ml.

20. The method of claim 14, wherein the endothelial cell is Human Umbilical Vein Endothelial Cells (HUVEC).

21. The method of claim 14, wherein the density of the endothelial cells within the graft is at least 40,000 per 200 μl culture.

22. The method of claim 14, wherein the viability of endothelial cells is at least 90%.

23. The method of claim 14, wherein no angiogenesis-inducing growth factors is added.

24. A 3-dimensional tissue graft comprising:
an ultrashort peptide scaffold, wherein the ultrashort peptide scaffold comprises at least one ultrashort peptide having a general formula selected from:
AₙBₘX, BₘAₙX, XAₙBₘ and XBₘAₙ
wherein the total number of amino acids of the ultrashort peptide does not exceed 7 amino acids;
wherein A is an aliphatic amino acids, selected from the group consisting of: isoleucine, leucine or any combination thereof, with n being an integer being selected from 0-5;
wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 0-3;
wherein X is comprised of a polar amino acid, selected from the group consisting of: aspartic acid, glutamic acid, lysine, arginine, histidine, cysteine, serine, threonine, asparagine, and glutamine; and
at least one endothelial cell,
wherein the endothelial cell forms network of tube-like structure.


SEQUENCE LISTING (4 sequences)

SEQ ID NO: 1 - protein, 4 aa, Artificial Sequence
  Synthetic
IIFK

SEQ ID NO: 2 - protein, 4 aa, Artificial Sequence
  Synthetic
  SITE (3)..(3): Xaa is cyclohexylalanine
IIXK

SEQ ID NO: 3 - protein, 4 aa, Artificial Sequence
  Synthetic
KFII

SEQ ID NO: 4 - protein, 4 aa, Artificial Sequence
  Synthetic
  SITE (2)..(2): Xaa is cyclohexylalanine
KXII